SEQUENCE LISTING (6 sequences)

SEQ ID NO: 1 - DNA, 1758 bp, Brassica napus
actttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgcgttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaattggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtactga

SEQ ID NO: 2 - DNA, 1758 bp, Brassica napus
actttcgtctcccgctacgctcccgacgagccccgcaagggtgctgatatcctcgtcgaagccctcgagcgtcaaggcgtcgaaaccgtctttgcttatcccggaggtgcttccatggagatccaccaagccttgactcgctcctccaccatccgtaacgtccttccccgtcacgaacaaggaggagtcttcgccgccgagggttacgctcgttcctccggcaaaccgggaatctgcatagccacttcgggtcccggagctaccaacctcgtcagcgggttagcagacgcgatgcttgacagtgttcctcttgtcgccattacaggacaggtccctcgccggatgatcggtactgacgccttccaagagacaccaatcgttgaggtaacgaggtctattacgaaacataactatttggtgatggatgttgatgacatacctaggatcgttcaagaagctttctttctagctacttccggtagacccggaccggttttggttgatgttcctaaggatattcagcagcagcttgcgattcctaactgggatcaacctatgcgcttacctggctacatgtctaggttgcctcagcctccggaagtttctcagttaggtcagatcgttaggttgatctcggagtctaagaggcctgttttgtacgttggtggtggaagcttgaactcgagtgaagaactggggagatttgtcgagcttactgggatccccgttgcgagtactttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggctttcgctagcagggctaaaattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttccctttgagcttcaaaacgtttggagaagccattcctccgcagtacgcgattcagatcctcgacgagctaaccgaagggaaggcaattatcagtactggtgttggacagcatcagatgtgggcggcgcagttttacaagtacaggaagccgagacagtggctgtcgtcatcaggcctcggagctatgggttttggacttcctgctgcgattggagcgtctgtggcgaaccctgatgcgattgttgtggatattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacaccaggaccatacctgttggatgtgatatgtccgcaccaagaacatgtgttaccgatgatcccaaatggtggcactttcaaagatgtaataacagaaggggatggtcgcactaagtactga

SEQ ID NO: 3 - protein, 585 aa, Brassica napus
TFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQLEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 4 - protein, 585 aa, Brassica napus
TFVSRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQILDELTEGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPNGGTFKDVITEGDGRTKY

SEQ ID NO: 5 - DNA, 1959 bp, Brassica
atggcggcggcaacatcgtcttctccgatctccttaaccgctaaaccttcttccaaatcccctctacccatttccagattctcccttcccttctccttaaccccacagaaaccctcctcccgtctccaccgtccactcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgcgttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaattggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtactga

SEQ ID NO: 6 - protein, 652 aa, Brassica
MAAATSSSPISLTAKPSSKSPLPISRFSLPFSLTPQKPSSRLHRPLAISAVLNSPVNVAPEKTDKIKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQLEDRFYKANRAHTYLGDPARENEIFPNMLQFAGACGIPAARVTKKEELREAIQTMLDTPGPYLLDVICPHQEHVLPMIPSGGTFKDVITEGDGRTKY

(19) United States
(12) Patent Application Publication
(10) Pub. No.: US 2014/0082772 A1
(43) Pub. Date: Mar. 20, 2014

(54) Herbicide-Tolerant Plants

(71) Applicants: Herve Vantieghem, Stutensee (DE); Matthias Pfenning, Schwegenheim (DE); Hagen Bremer, Ludwigshafen (DE); Ron Kehler, Mississauga (CA); Alfons Schoenhammer, Limburgerhof (DE)

(72) Inventors: Herve Vantieghem, Stutensee (DE); Matthias Pfenning, Schwegenheim (DE); Hagen Bremer, Ludwigshafen (DE); Ron Kehler, Mississauga (CA); Alfons Schoenhammer, Limburgerhof (DE)

(21) Appl. No.: 13/635,154

(22) PCT Filed: Mar. 17, 2011
(86) PCT No.: PCT/IB2011/000704

§ 371 Date: May 10, 2013

Related U.S. Application Data
(60) Provisional application No. 61/314,901, filed on Mar. 17, 2010.
(60) Provisional application No. 61/410,802, filed on Nov. 5, 2010.
(60) Provisional application No. 61/417,132, filed on Nov. 24, 2010.

(51) Int. Cl. C12N 15/82

(52) U.S. Cl. CPC C12N 15/8278
USPC 800/300; 504/212; 504/241; 504/215; 504/273; 504/213; 504/206; 47/58.1 SE

(57) ABSTRACT

The present invention provides herbicide-tolerant winter-type Brassica plants. The present invention also provides methods for controlling the growth of weeds by applying an herbicide to which herbicide-tolerant plants of the invention are tolerant. Plants of the invention express an AHAS enzyme that is tolerant to the action of one or more AHAS enzyme inhibitors.

### CROSS-REFERENCE TO RELATED APPLICATIONS

This application claims priority to U.S. Provisional Applications for Patent Ser. Nos. 61/314,901 filed Mar. 17, 2010; 61/410,802 filed Nov. 5, 2010; and 61/417,132 filed Nov. 24, 2010, the disclosures of which are incorporated herein by reference in their entirety.

### BACKGROUND OF THE INVENTION

Acetohydroxyacid synthase (AHAS; EC 4.1.3.18) is the first enzyme that catalyzes the biochemical synthesis of the branched chain amino acids valine, leucine, and isoleucine (Singh B. K., 1999 Biosynthesis of valine, leucine, and isoleucine in: Singh B. K. (Ed) Plant amino acids. Marcel Dekker Inc. New York, N.Y. Pg 227-247). AHAS is the site of action of four structurally diverse herbicide families including the sulfonylureas (LaRossa R A and Falco S C, 1984 Trends Biotechnol. 2:158-161), the imidazolinones (Shaner et al., 1984 Plant Physiol. 76:545-546), the triazolopyrimidines (Subramanian and Gerwick, 1989 Inhibition of acetolactate synthase by triazolopyrimidines in (ed) Whitaker J R, Sonnet P E Biocatalysis in agricultural biotechnology. ACS Symposium Series, American Chemical Society. Washington, D.C. Pg 277-288), and the pyrimidinylbenzoates (Subramanian et al., 1990 Plant Physiol 94: 239-244) Imidazolinone (IMI) and sulfonylurea (SU) herbicides are widely used in modem agriculture due to their effectiveness at very low application rates and relative non-toxicity in animals. By inhibiting AHAS activity, these families of herbicides prevent further growth and development of susceptible plants including many weed species.

Imidazolinone-tolerant canola has been developed through mutagenesis and selection with imidazolinone herbicides (S. Tan et al, Pest Management Science 61, 2005, 246). Commercial varieties were developed on the basis of the two most tolerant mutants, PM1 and PM2, and are currently marketed under the Clearfield® trademark. PM1 is known to be tolerant to imidazolinones only, whereas PM2 is cross-tolerant to both imidazolinones and sulfonylureas.

Although a PM2 mutant gene can provide some level of tolerance to imidazolinone and/or sulfonylurea herbicides, those oilseed rape (OSR) plants reported to date that contain a single PM2 mutant gene have exhibited insufficient tolerance to SU herbicides. For example, thifensulfuron application to the PM-mutant gene-containing, spring-type B. napus cultivar, 45A77, was shown to lead to reduced canola biomass, herbicide injury symptoms, or delayed maturity (R. Degenhardt et al., Weed Technology 19, 2005, 608).

In addition, there are four commercially available winter oilseed rape (WOSR) lines that are recognized in the art to provide tolerance to soil residues of sulfonylurea herbicides that are present as carry-over from prior wheat or pea crops. These four are B. napus variety ‘Sumner’ from Kansas State University; Roundup Ready lines DKW46-15 and DKW47-15 from DeKalb; and Roundup Ready line HyClass 115W from Croplan Genetics.

However, carry-over tolerant WOSR plants have been found to exhibit limited tolerance to soil SU herbicide residues present above carryover levels, i.e., above a residual concentration of about 0.5×, remaining from the applied herbicide dose. Thus, there remains a need in the art for winter-type Brassica plants that exhibit tolerance to greater than carryover levels of sulfonylurea herbicide.

### BRIEF SUMMARY OF THE INVENTION

The present invention provides herbicide-tolerant (HT) winter-type Brassica plants expressing one or more herbicide-tolerant AHASL genes and methods employing such plants. HT winter-type Brassica plants of the invention, containing one or fewer AHASL gene encoding a PM2 or similar mutations, unexpectedly exhibit no significant injury when contacted with an amount of SU herbicide that typically causes a non-tolerant plant to exhibit significant injury. For example, when contacted with a 1× rate of SU herbicide, on a scale from 1 to 10, with 1 indicating no visible damage and 10 indicating death of the plant, an HT Brassica plant hereof exhibits a score of 1.

The present invention is based on an unexpected discovery that winter-type Brassica crops (as exemplified by winter oilseed rape (WOSR), i.e., winter-type B. napus canola) that contains a mutant AHAS gene(s) providing imidazolinone tolerance, or that contain a mutant AHAS gene that normally provides insufficient SU herbicide tolerance, e.g., in spring-type OSR, surprisingly exhibit a commercially useful level of tolerance to certain sulfonylurea herbicides, i.e., a subgroup of the sulfonylurea herbicides. Yet, when the same genes are present in spring-types of the same Brassica crops, even if they do provide a commercially useful level of imidazolinone herbicide tolerance, the crops are found to be susceptible to these commercial levels of sulfonylurea herbicides.

The surprisingly high level of SU-herbicide tolerance exhibited by winter-type Brassica plants of the invention can occur when an herbicide-tolerant AHASL gene of interest is present in the Brassica A genome, preferably where such HT-AHASL gene is a variant of and is located at the plant's A genome native AHASL locus. In some embodiments, such an HT-AHASL is one obtained by mutagenesis, such as random mutagenesis of a Brassica A genome AHASL. When the herbicide-tolerant AHASL gene of interest is present solely in a genome other than the Brassica A genome, winter-type Brassica plants are found to be susceptible to the commercial levels of such SU-herbicides. Spring-types of the same Brassica crops having the herbicide-tolerant AHASL gene of interest in the Brassica A genome are also found to be susceptible to such levels of SU-herbicides.

HT winter-type Brassica plants of the present invention can include Brassica plants having at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica plant. Winter-type Brassica plants of the present invention having such a mono-SU-HT-AHASL gene can further contain in any genome thereof a second HT-AHASL gene encoding no P197X or W574X substitutions, but encoding a different HT substitution, such as an Other HT mutation. For example, winter-type Brassica plants of the present invention having an HT-AHASL gene encoding W574L homozygously, hemizygously or heterozygously in the A genome can also have a second HT-AHASL gene in the Brassica C genome, e.g., an AHAS gene encoding a S653N mutation.

The present invention provides methods of employing such HT winter-type Brassica plants including methods for controlling weeds, methods for selecting HT winter-type Brassica plants, and methods for providing yield protection for a winter-type Brassica crop. These methods can include performing post-emergent treatment or pre-emergent herbicide treatment of HT winter-type Brassica plants of the invention.

### BRIEF DESCRIPTION OF THE DRAWINGS

FIG. 1 provides a partial nucleotide sequence (SEQ ID NO:1) of a B. napus AHASL gene encoding the PM2 mutation (BnAHASL1A_PM2).

FIG. 2 provides a partial nucleotide sequence (SEQ ID NO:2) of a B. napus AHASL encoding the PM1 mutation (BnAHASL1C_PM1).

FIG. 3 provides a partial amino acid sequence (SEQ ID NO:3) of a B. napus AHASL gene having the PM2 mutation (BnAHASL1A_PM2).

FIG. 4 provides a partial amino acid sequence (SEQ ID NO:4) of B. napus AHASL having the PM1 mutation (BnAHASL1C_PM1).

FIG. 5 provides a second nucleotide sequence (SEQ ID NO:5) of a Brassica AHASL gene encoding the PM2 mutation (AHASL1A_PM2).

FIG. 6 provides a second amino acid sequence (SEQ ID NO:6) of a Brassica AHASL having the PM2 mutation (AHASL1A_PM2).

FIG. 7 is a graph showing AHAS enzyme activity in the presence of an imidazolinone herbicide.

FIG. 8 is a graph showing AHAS enzyme activity in the presence of a sulfonylurea herbicide.

### DETAILED DESCRIPTION OF THE INVENTION

### Definitions

As used herein, standard one letter abbreviations for amino acids will be used, for example, A indicates alanine, P indicates proline, W indicates tryptophan, X indicates any amino acid, etc. Mutations as compared to the wild-type sequence will be indicated by specifying the wild-type amino acid and position followed by the amino acid present in the mutant. For example, P197X will be used to indicate that the proline at position 197 can be substituted with any amino acid.

As used herein, the amino acid positions refer to the polypeptide of the large subunit of the plastidic, Brassica AHAS enzymes (AHASL). Amino acid positions in a Brassica AHASL referred to herein are numbered according to the industry standard numbering of residues corresponding to those in the Arabidopsis thaliana(At) AHASL sequence, and can be denoted with an (At). For example, P197(At) refers to the proline residue at the position in a Brassica AHASL that corresponds to the proline at position 197 of the Arabidopsis thaliana AHASL.

As used herein, “tolerant” or “herbicide-tolerant” indicates a plant or portion thereof capable of growing in the presence of an amount of herbicide that normally causes growth inhibition in a non-tolerant (e.g., a wild-type) plant or portion thereof. Levels of herbicide that normally inhibit growth of a non-tolerant plant are known and readily determined by those skilled in the art. Examples include the amounts recommended by manufacturers for application. The maximum rate is an example of an amount of herbicide that would normally inhibit growth of a non-tolerant plant.

As used herein, “herbicide tolerant (HT) AHASL” refers to the AHASL polypeptide expressed from one HT AHASL allele of an AHASL gene in a plant cell and/or from either or both of two homologous alleles of the same HT AHASL gene, i.e., in the same genome of the plant cell, whereby the HT-AHASL can provide herbicide tolerance to an AHAS enzyme of the plant cell. An HT-AHASL gene can be recombinant, or can be obtained by application of a mutagenesis process, a breeding process, or other process known in the art. Such a gene can be hemizygous, heterozygous, or homozygous.

As used herein, “AHAS” and “AHASL” respectively refer to functional, plastidic AHAS enzymes and AHASL polypeptides thereof, i.e., which are functional in cells of the Brassica plants as described herein. Similarly, teams such as “gene” and “polynucleotide”, when used in reference to those encoding such an “AHAS” and “AHASL,” refer to functional genes therefor, i.e., genes that are expressible in such a cell.

As used herein in regard to herbicides useful in various embodiments hereof, terms such as AHAS inhibitor, ACCase inhibitor, PPO inhibitor, EPSPS inhibitor, imidazolinone, sulfonylurea, and the like, refer to those agronomically acceptable herbicide active ingredients (A.I.) recognized in the art. Similarly, teams such as fungicide, nematicide, pesticide, and the like, refer to other agronomically acceptable active ingredients recognized in the art.

When used in reference to a particular mutant enzyme or mutant polypeptide thereof, terms such as herbicide tolerant (HT) and herbicide tolerance refer to the ability of such enzyme (or the ability of the polypeptide to confer to its enzyme the ability) to tolerate an herbicide A.I. that would normally inactivate or inhibit the activity of the wild-type (non-mutant) version of said enzyme. When used specifically in regard to an AHAS enzyme, or AHASL polypeptide, it refers specifically to the ability to tolerate an AHAS-inhibitor. Classes of AHAS-inhibitors include sulfonylureas, imidazolinones, triazolopyrimidines, sulfonylaminocarbonyltriazolinones, and pyrimidinylbenzoates.

As used herein, “recombinant” refers to an organism having genetic material from different sources as a result of human application of a recombinogenic technique.

As used herein, “mutagenized” refers to an organism having an altered genetic material as compared to the genetic material of a corresponding wild-type organism, wherein the alteration(s) in genetic material were induced and/or selected by human action. Examples of human action that can be used to produce a mutagenized organism include, but are not limited to, tissue culture of plant cells (e.g., calli) in sub-lethal concentrations of herbicides (e.g., sulfonylurea herbicides), treatment of plant cells with a chemical mutagen and subsequent selection with herbicides (e.g., sulfonylurea herbicides); or by treatment of plant cells with x-rays and subsequent selection with herbicides (e.g., sulfonylurea herbicides). Any method known in the art can be used to induce mutations. Methods of inducing mutations can induce mutations in random positions in the genetic material or can induce mutations in specific locations in the genetic material (i.e., can be directed mutagenesis techniques).

As used herein, a “genetically modified organism” (GMO) is an organism whose genetic characteristics have been altered by human effort causing insertion of genetic material from another source organism or progeny thereof that retain the inserted genetic material. The source organism can be of a different type of organism (e.g., a GMO plant can contain bacterial genetic material) or from the same type of organism (e.g., a GMO plant can contain genetic material from another plant). As used herein, recombinant and GMO are considered synonyms and indicate the presence of genetic material from a different source whereas mutagenized indicates altered genetic material from a corresponding wild-type organism but no genetic material from another source organism.

As used herein, “wild-type” or “corresponding wild-type plant” means the typical form of an organism or its genetic material, as it normally occurs, as distinguished from, e.g., mutagenized and/or recombinant forms.

As used herein, an herbicide-tolerance-inducing mutation “HT-mutation” is an alteration in the amino acid sequence of an AHASL enzyme that confers tolerance to one or more herbicides (i.e., sulfonylurea herbicides, imidazolinone herbicides, etc).

In some embodiments, an HT-mutation can be an “SU-HT-mutation”, i.e., a mutation selected from the group consisting of P197X and W574X. In some embodiments, an SU-HT-mutation can be selected from the group consisting of P197S, P197A, P197E, P197L, P197Q, P197R, P197S, P197V, P197W, P197Y, P197I, P197H, P197C, and P197G. In other embodiments, an SU-HT-mutation can be selected from the group consisting of P197S, P197L, and P197T. In some embodiments, an SU-HT-mutation can be selected from the group consisting of W574L, W574M, W574C, W574S, W574R, W574G, W574A, W574F, W574Q, and W574Y. In some embodiments, an SU-HT-mutation can comprise W574L.

In some embodiments, an HT-mutation can be an “Other HT-mutation”. As used herein, an “Other HT-mutation” is an alteration in the amino acid sequence of an AHASL enzyme that confers tolerance to one or more herbicides (i.e., sulfonylurea herbicides, imidazolinone herbicides, etc) wherein the alteration is at a position other than proline 197 or tryptophan 574. The following Table 1 provides a list of possible sites for Other HT-mutations, permissible substitutions, preferred substitutions, and more preferred substitutions. X indicates any amino acid.

TABLE 1
“Other” HT Mutations

| w/t (At) | Permissible Sub. | Pref. Sub. | More. Pref. |
|---|---|---|---|
| G121 | X | N SAD | |
| A122 | X | TV DPY (or X) | TV |
| M124 | X | E I | |
| R142 | X | K | |
| V196 | X | M | |
| R199 | X | AE | AE |
| T203 | X | I | |
| A205 | X | V CDERTWYN | V |
| F206 | X | RAHWY | |
| K256 | X | DENPTG | |
| M351 | X | CKVGPQY | |
| H352 | X | FMQ | |
| R373 | X | F | |
| D375 | X | NAE | |
| D376 | X | EVN GPSWAC | |
| R377 | X | K | |
| M570 | X | ANC | |
| V571 | X | ACNYIQSW | |
| F578 | X | CGLNRDEIKPSW | |
| S653 | X | N IFT | N |
| G654 | X | QCED | E |

In some embodiments, Other HT-mutations can be selected from the group consisting of A122X, R199X, A205X, S653X, and G654X, and combinations thereof. In other embodiments, Other HT-mutations can be selected from the group consisting of A122T, A122V, A122D, A122P, A122Y, R199A, R199E, A205V, A205C, A205D, A205E, A205R, A205T, A205W, A205Y, A205N, S653N, S653I, S653F, S653T, G654Q, G654C, G654E, G654D, and combinations thereof. In some embodiments, Other HT-mutations can be selected from the group consisting of A122T, A122V, R199A, R199E, A205V, S653N, G654E, and combinations thereof.

Plants

Sources of useful plastidic AHASL genes can be provided from any of the following deposited cell lines listed in Table 2, of Brassica napus (Bn) and Brassica juncea (Bj), wherein their AHAS-inhibitor-tolerant (HT) AHAS large subunit (AHASL) alleles are referred to as shown below, with the final letter indicating the Brassica genome (A, B, or C) to which the allele is native: BnAHASL1A or BnAHASL1C for B. napus, and BjAHASL1A or BjAHASL1B for B. juncea. Note that AHASL mutation positions are stated with reference to the standardized nomenclature in the field, in which the Arabidopsis thaliana (At) plastidic AHASL polypeptide provides the standard for residue position numbering.

TABLE 2
Examples of Brassica Line Sources for Useful AHASL Genes

| | | | | |
|---|---|---|---|---|
| Name of | | ATCC | AHAS | |
| Line | Species | Deposit | Allele | Mutation |

U.S. Pat. No. 5,545,821 to Wong et al.

| | | | | |
|---|---|---|---|---|
| PM-1 | B. napus | 40683 | BnAHASL1C | S653(At)N |
| PM-2 | B. napus | 40684 | BnAHASL1A | W574(At)L |

PCT Application No. PCT/US09/58169 to Beetham et al.

| | | | | |
|---|---|---|---|---|
| BnCL120C7 | B. napus | PTA-9278 | BnAHASL1A | A122(At)T |
| BnCL131A1 | B. napus | PTA-9279 | BnAHASL1A | A122(At)T + |
| | | | | S653(At)N |
| BnCL140B3 | B. napus | PTA-9402 | BnAHASL1A | A122(At)T + |
| | | | | S653(At)N |
| BnCL140C7 | B. napus | PTA-9403 | BnAHASL1A | A122(At)T + |
| | | | | S653(At)N |
| PM1PM2/ | B. napus | PTA-10321 | BnAHASL1C | S653(At)N |
| CL131A1 | | | | |
| | | | BnAHASL1A | W574(At)L |
| | | | BnAHASL1A | A122(At)T + |
| | | | | S653(At)N |

U.S. Pat. No. 7,355,098 to Yao et al.

| | | | | |
|---|---|---|---|---|
| J04E-0044 | B. juncea | PTA-6324 | BjAHASL1B | S653(At)N |
| J04E-0122 | B. juncea | PTA-7944 | BjAHASL1A | A122(At)T |
| J04E-0130 | B. juncea | PTA-7945 | BjAHASL1B | A122(At)T |
| J04E-0139 | B. juncea | PTA-7946 | BjAHASL1A | S653(At)N |

PCT Publication WO 2009/031031 to Yao et al.

| | | | | |
|---|---|---|---|---|
| J05Z-07801 | B. juncea | PTA-8305 | BjAHASL1B | S653(At)N |
| | | | BnAHASL1A | W574(At)L |

Patents documents referred to in Table 2 are hereby incorporated in their entirety. As is WO 2009/046334 Schopke et al.

Although exemplified with reference to winter-type, AHAS-inhibitor-tolerant Brassica napus Canola/OSR varieties, it is believed that in various embodiments, the presently described methods using sulfonylurea herbicides can be employed with other commercially valuable, winter-type, AHAS-inhibitor-tolerant Brassica species, such as B. oleracea, B. rapa, B. nigra, and B. juncea. AHAS-inhibitor-tolerant Brassica lines described as useful herein can be employed in the weed control methods either directly or indirectly, i.e., either as crops for herbicide treatment or as AHAS-inhibitor-tolerance trait donor lines for development, as by traditional plant breeding, to produce other winter-type Brassica varietal and/or hybrid crops containing such trait or traits. All such resulting variety or hybrids crops, containing the ancestral AHAS-inhibitor-tolerance trait or traits can be referred to herein as progeny of the ancestral, AHAS-inhibitor-tolerant line(s). In the case of Brassica A-, B-, and C-genome AHASL traits, these can be bred into winter-type Brassica species having a corresponding genome, e.g.: B. napus (AACC), B. juncea. (AABB), B. oleracea (CC), B. rapa (AA), B. nigra (BB), B. carinata (BBCC), and Raphanobrassica varieties that are progeny of a cross between any of the foregoing and a Raphanus spp., e.g., Raphanobrassica var. ‘rabbage’ (RRCC) from B. oleracea×Raphanus sativus or Raphanobrassica var. ‘raparadish’ (RRAA) from B. rapa×Raphanus sativus. Among these, B. napus, B. rapa, and B. juncea are of particular interest, with B. napus being preferred in some embodiments.

Plants of the invention include those plants which, in addition to having been rendered sulfonylurea-tolerant, have been subjected to further genetic modifications by breeding, mutagenesis or genetic engineering, e.g. have been rendered tolerant to applications of specific other classes of herbicides, such as auxin herbicides, dicamba or 2,4-D; bleacher herbicides such as hydroxyphenylpyruvate dioxygenase (HPPD) inhibitors or phytoene desaturase (PDS) inhibitors; enolpyruvyl shikimate 3-phosphate synthase (EPSP) inhibitors such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i.e., bromoxynil or ioxynil) herbicides as a result of conventional methods of breeding or genetic engineering; furthermore, sulfonylurea-tolerant winter oilseed rape (winter canola) can have been made resistant to multiple classes of herbicides through multiple genetic modifications, such as resistance to both glyphosate and glufosinate or to both glyphosate and a herbicide from another class such as HPPD inhibitors, auxin herbicides, or ACCase inhibitors. These herbicide resistance technologies are, for example, described in Pest Management Science at volume, year, page 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein.

Furthermore, sulfonylurea-tolerant winter oilseed rape (winter canola) is also covered which is by the use of recombinant DNA techniques capable to synthesize one or more insecticidal proteins, especially those known from the bacterial genus Bacillus, particularly from Bacillus thuringiensis, such as S-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such streptomycete toxins; plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda).

Furthermore, sulfonylurea-tolerant winter oilseed rape (winter canola) is also covered which is by the use of recombinant DNA techniques capable to synthesize one or more proteins to increase the resistance or tolerance of those plants to bacterial, viral or fungal pathogens. The methods for producing such genetically modified plants are generally known to the person skilled in the art.

Furthermore, sulfonylurea-tolerant winter oilseed rape (winter canola) is also covered which is by the use of recombinant DNA techniques capable to synthesize one or more proteins to increase the productivity (e.g. oil content), tolerance to drought, salinity or other growth-limiting environmental factors or tolerance to pests and fungal, bacterial or viral pathogens of those plants.

Furthermore, sulfonylurea-tolerant winter oilseed rape (winter canola) is also covered which contains by the use of recombinant DNA techniques a modified amount of substances of content or new substances of content, specifically to improve human or animal nutrition, e.g. oil crops that produce health-promoting long-chain omega-3 fatty acids or unsaturated omega-9 fatty acids (e.g. Nexera® rape, Dow Agro Sciences, Canada).

In some embodiments, the present invention provides agronomic products, for example, seed oil, seed meal, and the like. In some embodiments, said agronomic products can be of feed quality or food quality. The agronomic products can be produced from plants, including seeds of said plants, treated by or obtained from the methods described throughout the detailed description herein.

AHAS Enzymes

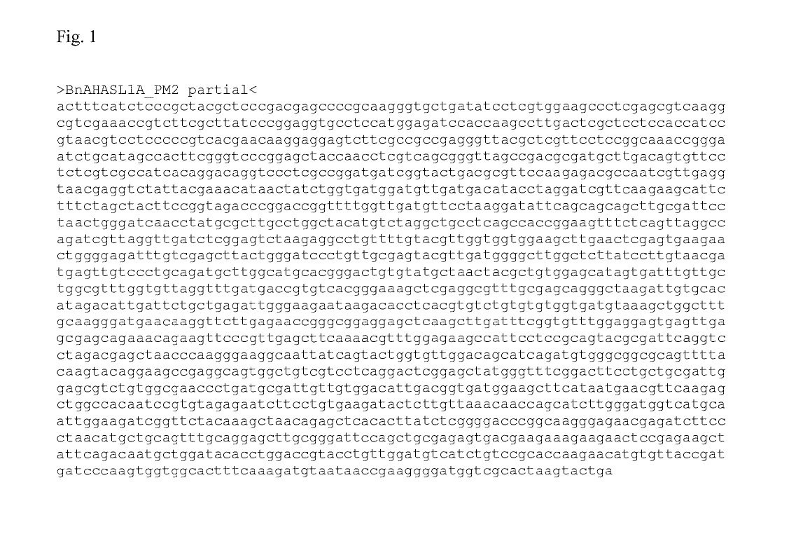

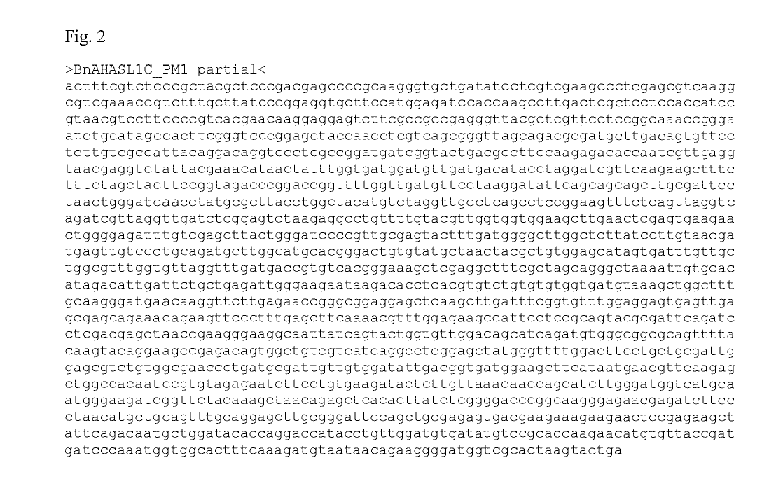

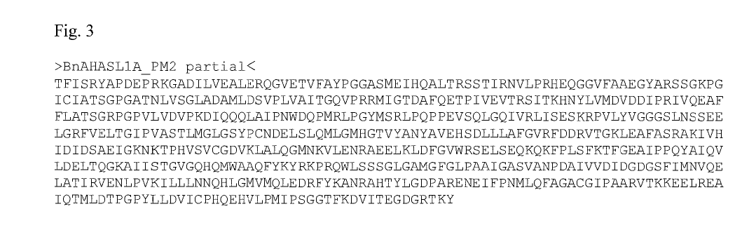

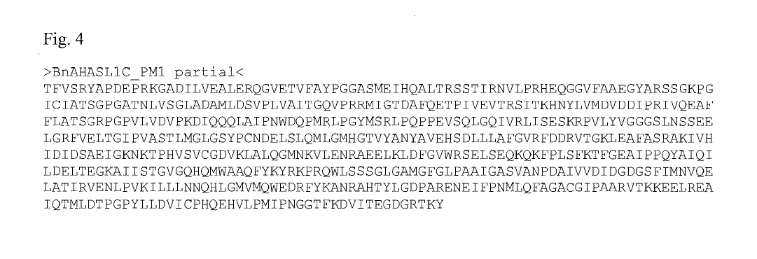

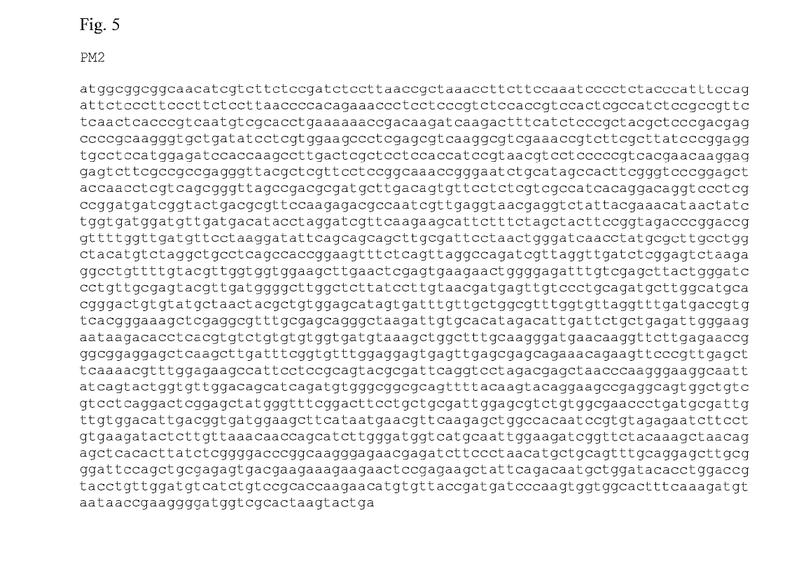

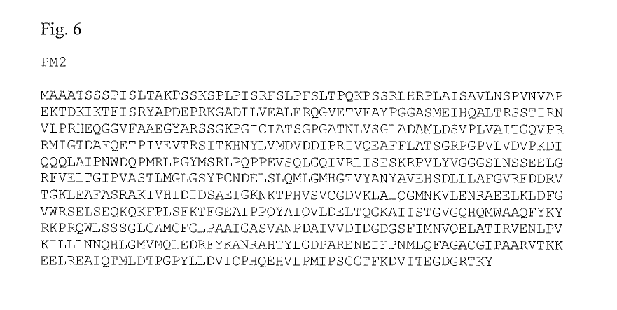

In various embodiments, winter-type Brassica plants containing both a W574(At)X and a S653(At)X in plastidic AHASL polypeptides thereof can be used. These can be present in different alleles, such as on different genomes, with each containing a single mutation in the respective AHASL gene, or these two can be present in a single AHASL, as a double-mutant allele. In various embodiments, these can be W574(At)L and S653(At)N: the former can be referred to as the “PM2” mutation and the latter as the “PM1” mutation. FIG. 1 (SEQ ID NO: 1) and FIG. 3 (SEQ ID NO:3) provide a partial nucleotide sequence and partial amino acid sequence, respectively, for the PM2 mutation in B. napus. FIG. 2 (SEQ ID NO:2) and FIG. 4 (SEQ ID NO:4) provide a partial nucleotide sequence and partial amino acid sequence, respectively, for the PM1 mutation in B. napus. FIG. 5 (SEQ ID NO:5) and FIG. 6 provide a second nucleotide sequence and second amino acid sequence, respectively, for the PM2 mutation, for example the PM2 sequence introgressed into B. juncea from B. napus.

The winter-type Brassica plants hereof can be inbred varieties, e.g., open-pollinated varieties, or hybrids, e.g., F1 hybrids.

Although transgenic or non-transgenic mutant AHAS traits can be employed herein, in winter-type Brassica crops, in various embodiments, the trait or traits can be non-transgenic, i.e., obtained by a process, excluding recombinant DNA techniques, and comprising mutagenesis, genoplasty, and/or isolation of spontaneous mutant plants. Many mutagenesis techniques are known in the art and these can involve application of a mutagenic chemical agent or radiation to seeds, plants parts, or cultured plant cells; alternatively, or in addition, the culturing of plant cells, or the conditions under which plant cells are cultured, can increase the rate of occurrence or accumulation of spontaneous mutations. Genoplasty techniques can include directed mutation-type strategies, such as methods comprising introduction, into the plant cell nucleus, of oligonucleotides that facilitate mismatch-repair-system-mediated nucleotide substitution.

Note that, in AHAS enzymes, there are two mutation sites known to be amenable to mutations that provide significant levels of tolerance to SUs. These both occur in AHASL at positions P197(At) and W574(At).

In various embodiments, the WOSR and other winter-type Brassica crops can contain one such mutation in the plastidic AHASL(s) thereof; in addition to one or more other mutations, in the same or different plastidic AHASL gene, that can be selected from those at sites where mutations have been found to be capable of providing tolerance toward one or more other AHAS inhibitor, examples of which sites include G121(At), A122(At), M124(At), R142(At), V196(At), R199(At), T203(At), A205(At), F206(At), K256(At), M351(At), H352(At), R373(At), D375(At), D376(At), R377(At), M570(At), V571(At), F578(At), S653(At), and G654(At).

Thus, in some embodiments, the WOSR and other winter-type Brassica crops useful herein can contain one SU-HT mutation in the plastidic AHASL(s) thereof. In various embodiments, a plant useful herein can contain more than one herbicide tolerance mutation in the plastidic AHASL population therein. In various embodiments, these can be contained on different, single-mutant AHASL genes.

In sum, a WOSR or other winter-type Brassica crop useful herein can contain up to one expressible plastidic AHASL gene that encodes a mutation at P197(At) or W574(At), whether or not that AHASL gene also encodes other AHAS-inhibitor-tolerance mutation(s) [i.e., other than any additional mutation at a position selected from P197(At) or W574(At)], and whether or not that gene is represented by a single allele, as a heterozygote, or by two alleles, as a homozygote. Such WOSR or other Brassica crop does not contain more than one plastidic AHASL gene that encodes a mutation selected from those occurring at positions P197(At) or W574(At) in the A genome.

When the WOSR or other winter-type Brassica crop contains an expressible plastidic AHASL gene encoding a mutation at P197(At) or W574(At), in a Brassica A-genome allele, then no such additional mutation is required to be present in an AHASL gene of the plant. Such embodiments would not include winter-type crops of B. oleracea (CC), B. nigra (BB), B. carinata (BBCC), and Raphanobrassica var. ‘rabbage’ (RRCC), which lack a Brassica A-genome.

As described herein, plants useful in various embodiments hereof contain one or more mutant AHASL gene(s) wherein at least one mutation therein confers herbicide tolerance to the AHAS enzyme of which the encoded AHASL is a part, and thereby confers herbicide tolerance to the plant in which it resides. Such a mutant AHASL gene is referred to as an “HT-AHASL gene”. Also as described herein, plants useful in various embodiments hereof can contain, as one such HT-AHASL gene, an SU-HT-AHASL gene, i.e., an HT-AHASL gene encoding a mutation selected from among P197X and W574X, such P197X and W574X mutations being referred to herein as sulfonylurea-tolerance HT mutations or “SU-HT” mutations. Plants hereof can contain only one such SU-HT-AHASL gene, and this can be a “mono-SU-HT-AHASL” gene. Said mono-SU-HT-AHASL gene can be located in the Brassica A genome. As used herein, a “mono-SU-HT-AHASL” gene refers to an HT-AHASL gene that encodes only one SU-HT mutation, or only one SU-HT mutation per allele of said one gene. Thus, a “mono-SU-HT-AHASL” gene refers to an HT-AHASL gene that:

(1) encodes
(a) only one of P197X or W574X and encodes it homozygously or hemizygously;
(b) only one of P197X or W574X and encodes it in one allele and encodes a wild-type P197P or W574W residue, respectively, in the homologous allele;
(c) two different P197X mutations heterozygously and is homozygous for wild-type W574W residues;
(d) two different W574X mutations heterozygously and is homozygous for wild-type P197P residues; or
(e) both (i) one P197X mutation and a wild-type W574W residue in one allele, and (ii) one W574X mutation and a wild-type P197P residue in the homologous allele; and
   (2) optionally encodes Other HT mutation(s), as that term is defined herein.
   In some embodiments, a mono-SU-HT-AHASL gene can encode no Other HT mutations.

The term “hemizygous” when used herein in regard to an AHASL mutation's being encoded “hemizygously” refers to the relationship between the corresponding loci of two homologous chromosomes in a genome, wherein one of the two loci is occupied by a (functioning) AHASL allele that contains the amino acid residue of the (substitution) mutation and the other locus either is occupied by a non-functioning AHASL allele or is unoccupied, e.g., the second allele being absent or having been deleted. Note that “mono-SU-HT-AHASL” genes listed under (1)(b)-(1)(e) above can be referred to as “heterozygous-mono-SU-HT-AHASL” genes, i.e., since they encode each SU-HT mutation heterozygously.

As described herein, Brassica plants in various embodiments hereof can contain at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica plant.

In addition to being able to tolerate herbicides that inhibit AHAS activity, plants of the invention may also be able to tolerate herbicides that work on other physiological processes. For example, plants of the invention may be tolerant to acetyl-Coenzyme A carboxylase (ACCase) inhibitors, such as “dims” (e.g., cycloxydim, sethoxydim, clethodim, or tepraloxydim), “fops” (e.g., clodinafop, diclofop, fluazifop, haloxyfop, or quizalofop), and “dens” (such as pinoxaden); to inhibitors of 5-enolpyruvylshikimate-3-phosphate synthase (EPSPS) such as glyphosate; to inhibitors of protoporphyrinogen [IX] oxidase (PPO) such as saflufenacil; and to inhibitors of glutamine synthetase such as glufosinate. In addition to these classes of inhibitors, plants of the invention may also be tolerant of herbicides having other modes of action, for example, auxin growth regulators (e.g., dicamba), chlorophyll/carotenoid pigment inhibitors, cell membrane destroyers, photosynthesis inhibitors, cell division inhibitors, root inhibitors, shoot inhibitors, and combinations thereof. Such tolerance traits may be expressed, e.g.: as mutant ACCase proteins, mutant EPSPS proteins, or mutant glutamine synthetase proteins; or as mutant native, inbred, or transgenic aryloxyalkanoate dioxygenase (AAD or DHT), haloarylnitrilase (BXN), 2,2-dichloropropionic acid dehalogenase (DEH), dicamba monooxygenase (DMO), glyphosate-N-acetyltransferase (GAT), glyphosate decarboxylase (GDC), glyphosate oxidoreductase (GOX), glutathione-S-transferase (GST), phosphinothricin acetyltransferase (PAT or bar), or cytochrome P450 (CYP450) proteins having an herbicide-degrading activity. Winter Brassica plants hereof can also be stacked with other traits including, but not limited to, pesticidal traits such as Bt Cry and other proteins having pesticidal activity toward coleopteran, lepidopteran, nematode, or other pests; nutrition or nutraceutical traits such as modified oil content or oil profile traits, high protein or high amino acid concentration traits, and other trait types known in the art.

The present invention also encompasses progeny of the plants of the invention as well as seeds derived from the herbicide-tolerant plants of the invention and cells derived from the herbicide-tolerant plants of the invention. The present invention also provides methods for producing seed by performing the methods described throughout the detailed description hereof and harvesting seed from the herbicide-tolerant plants. The present invention provides seed harvested from Brassica plants treated by methods described throughout the

### DETAILED DESCRIPTION

Nucleic Acid Molecules

The present invention also encompasses nucleic acid molecules that encode all or a portion of the AHASL proteins described above. Nucleic acid molecules of the invention can comprise a nucleic acid sequence encoding an amino acid sequence comprising a modified, or where applicable, unmodified, version of the sequences listed in the patent documents referenced in Table 2, wherein the resulting sequence encodes an AHASL protein that comprises one or more of the following: the amino acid at position 197 is other than proline while the amino acid at position 574 is tryptophan; or the amino acid at position 574 is other than tryptophan while the amino acid at position 197 is proline.

The present invention also encompasses nucleic acids that encode Brassica AHASLs having one or more Other HT-mutations. Such AHASLs can also comprise amino acid sequences having one or more of the following: the amino acid at position 197 is other than proline while the amino acid at position 574 is tryptophan; or the amino acid at position 574 is other than tryptophan while the amino acid at position 197 is proline.

A nucleic acid molecule of the invention can be DNA, derived from genomic DNA or cDNA, or RNA. A nucleic acid molecule of the invention can be naturally occurring or can be synthetic. A nucleic acid molecule of the invention can be isolated, recombinant and/or mutagenized.

Nucleic acid molecules of the invention can comprise non-coding sequences, which may or may not be transcribed. Non-coding sequences that can be included in the nucleic acid molecules of the invention include, but are not limited to, 5′ and 3′ UTRs, polyadenylation signals and regulatory sequences that control gene expression (e.g., promoters). Nucleic acid molecules of the invention can also comprise sequences encoding transit peptides, protease cleavage sites, covalent modification sites and the like. In one embodiment, nucleic acid molecules of the invention encode a chloroplast transit peptide sequence in addition to a sequence encoding an AHAS enzyme.

In another embodiment, nucleic acid molecules of the invention can encode an AHASL having at least 50%, 60%, 70%, 75%, 80%, 85%, 90%, 95% or more sequence identity to a P197X or W574X AIIASL as described above, wherein the protein encoded by the sequence comprises one or more of the following: the amino acid at position 197 is other than proline while the amino acid at position 574 is tryptophan; or the amino acid at position 574 is other than tryptophan while the amino acid at position 197 is proline.

As used herein, “percent (%) sequence identity” is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program BLAST available at http://blast.ncbi.nlm.nih.gov/Blast.cgi with search parameters set to default values.

The present invention also encompasses nucleic acid molecules that hybridize to nucleic acid molecules encoding an AHAS enzyme of the invention as well as nucleic acid molecules that hybridize to the reverse complement of nucleic acid molecules encoding an AHAS enzyme of the invention. In one embodiment, nucleic acid molecules of the invention comprise nucleic acid molecules that hybridize to a nucleic acid molecule encoding a P197X or W574X AHASL as described above, wherein the protein encoded by the sequence comprises one or more of the following: the amino acid at position 197 is other than proline while the amino acid at position 574 is tryptophan; or the amino acid at position 574 is other than tryptophan while the amino acid at position 197 is proline as well as nucleic acid molecules complementary to all or a portion of the coding sequences, or the reverse complement of such nucleic acid molecules under stringent conditions. The stringency of hybridization can be controlled by temperature, ionic strength, pH, and the presence of denaturing agents such as formamide during hybridization and washing. Stringent conditions that can be used include those defined in Current Protocols in Molecular Biology, Vol. 1, Chap. 2.10, John Wiley & Sons, Publishers (1994) and Sambrook et al., Molecular Cloning, Cold Spring Harbor (1989) which are specifically incorporated herein as they relate to teaching stringent conditions.

In one embodiment, nucleic acid molecules invention encompasses oligonucleotides that can be used as hybridization probes, sequencing primers, and/or PCR primers. Such oligonucleotides can be used, for example, to determine a codon sequence at a particular position in a nucleic acid molecule encoding an AHAS enzyme, for example, by allele specific PCR. Such oligonucleotides can be from about 15 to about 30, from about 20 to about 30, or from about 20-25 nucleotides in length.

Herbicides

Herbicide compositions of the invention comprise one or more SU herbicides selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof. In some embodiments the herbicide composition can further comprise a significant amount of no other SU. Optionally, herbicide compositions of the invention can further comprise A.I.(s) belonging to one or more additional classes of AHAS-inhibitor herbicides, e.g., imidazolinone herbicides, and/or one or more A.I. of other classes, e.g., agronomic fungicides, bactericides, algicides, nematicides, insecticides, and the like.

Each SU has its own recommended 1× dose rate. The 1× dose rates for SU active ingredients useful herein are shown below; these are also applicable to the salt or ester forms thereof.

TABLE 3
Sulfonylurea herbicides and application rates

| Sulfonylurea(s) | 1x (g/ha) |
|---|---|
| Flupyrsulfuron | 10 |
| Imazosulfuron | 25 |
| Thifensulfuron | 30 |
| Tribenuron | 30 |
| Amidosulfuron | 30 |
| Foramsulfuron | 35 |
| Iodosulfuron | 10 |
| Mesosulfuron | 7.5 |
| Nicosulfuron | 30 |
| Mesosulfuron + | 14.4 |
| Iodosulfuron (5:1 w/w) | |

Pre-emergent or pre-planting weed control methods useful in various embodiments hereof utilize >0.5× application rates of SU applied within about 30 days prior to emergence; in some embodiments, this can be >0.6×, >0.7×, >0.8×, >0.9×, or >1× of SU.

In addition, merely carry-over-tolerant WOSR plants have been found to lack tolerance to, or to exhibit insufficient tolerance, to post-emergent SU treatments. As a result, post-emergent weed control methods useful in various embodiments hereof utilize >0.25× application rates of SU; in some embodiments, this can be >0.3×, >0.4×, >0.5×, >0.6×, >0.7×, >0.8×, >0.9×, or >1× of SU.

Selection methods for herbicide tolerant winter Brassica plants also can be performed using these treatment method parameters, wherein no weeds are present in the immediate vicinity of the Brassica plant or its planting locus.

In either pre-emergent or post-emergent weed control methods hereof, the method can utilize 1×SU application rates with no significant injury to the plant; in some embodiments thereof, the application rate can exceed 1×SU; in some embodiments, the rate can be up to 4×SU, though more typically it will be about 2.5× or less, or about 2× or less. Where a combination of these SU active ingredients is employed, the herbicide application rate will preferably provide a summed rate that falls within the >0.5× to 4× or 0.25× to 4×SU range. For example, a 5:1 w/w combination of mesosulfuron and iodosulfuron having a 1× dose rate of 18 g/ha will, if applied at that rate, provide about 15 g/ha and 3 g/ha of these A.I.s, respectively: these are approximately 2× and 0.3× application rates, providing a summed rate of SU treatment of about 2.3×SU.

The herbicidal compositions hereof comprising a herbicide selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, can be used in any agronomically acceptable format. For example, these can be formulated as ready-to-spray aqueous solutions, powders, suspensions; as concentrated or highly concentrated aqueous, oily or other solutions, suspensions or dispersions; as emulsions, oil dispersions, pastes, dusts, granules, or other broadcastable formats. The herbicide compositions can be applied by any means known in the art, including, for example, spraying, atomizing, dusting, spreading, watering, seed treatment, or co-planting in admixture with the seed. The use forms depend on the intended purpose; in any case, they should ensure the finest possible distribution of the active ingredients according to the invention.

Where the optional A.I. includes an AHAS-inhibitor, this can be selected from: (1) the imidazolinones, i.e., imazamox, imazethapyr, imazapyr, imazapic, imazaquin, and imazamethabenz, preferably from imazamox, imazethapyr, imazapyr, and imazapic, preferably imazamox; (2) the pyrimidinylbenzoates, i.e., including the pyrimidinyloxybenzoates (e.g., bispyribac, pyriminobac, and pyribenzoxim) and the pyrimidinylthiobenzoates (e.g., pyrithiobac and pyriftalid); and (3) the sulfonamides, i.e., including the sulfonylaminocarbonyltriazolinones (e.g., flucarbazone and propoxycarbazone) and the triazolopyrimidines (e.g., cloransulam, diclosulam, florasulam, flumetsulam, metosulam, and penoxsulam). The agronomically acceptable salts and esters of the foregoing are also included, as are combinations thereof.

Where the optional A.I. includes an herbicide from a different class to which the winter Brassica plant(s) hereof would normally be susceptible, the winter Brassica plant to be used is selected from among those that further comprise a trait of tolerance to such herbicide. Such further tolerance traits can be provided to the plant by any method known in the art, e.g., including techniques of traditional breeding to obtain a tolerance trait gene by hybridization or introgression, of mutagenesis, of genoplasty, and/or of transformation. Such plants can be described as having “stacked” traits.

Sulfonylurea herbicidal active ingredients useful in various embodiments hereof include those listed in Table 4.

TABLE 4
Sulfonylurea Herbicide Active Ingredients

| SU A.I. | Example Salt or Ester | “Other SU” |
|---|---|---|
| amidosulfuron | | — |
| azimsulfuron | | Other |
| bensulfuron | bensulfuron-methyl | Other |
| chlorimuron | chlorimuron-ethyl | Other |
| chlorsulfuron | | Other |
| cinosulfuron | | Other |
| cyclosulfamuron | | Other |
| ethametsulfuron | ethametsulfuron-methyl | Other |
| ethoxysulfuron | | Other |
| flazasulfuron | | Other |
| flucetosulfuron | | Other |
| flupyrsulfuron | flupyrsulfuron-methyl-sodium | — |
| foramsulfuron | | — |
| halosulfuron | halosulfuron-methyl | Other |
| imazosulfuron | | — |
| iodosulfuron | iodosulfuron-methyl-sodium | — |
| mesosulfuron | mesosulfuron-methyl | — |
| metazosulfuron | | Other |
| metsulfuron | metsulfuron-methyl | Other |
| nicosulfuron | | — |
| orthosulfamuron | | Other |
| oxasulfuron | | Other |
| primisulfuron | primisulfuron-methyl | Other |
| propyrisulfuron | | Other |
| prosulfuron | | Other |
| pyrazosulfuron | pyrazosulfuron-ethyl | Other |
| rimsulfuron | | Other |
| sulfometuron | sulfometuron-methyl | Other |
| sulfosulfuron | | Other |
| thifensulfuron | thifensulfuron-methyl | — |
| triasulfuron | | Other |
| tribenuron | tribenuron-methyl | — |
| trifloxysulfuron | trifloxysulfuron-sodium | Other |
| triflusulfuron | triflusulfuron-methyl | Other |
| tritosulfuron | | Other |

In some embodiments, an herbicide composition hereof that comprises a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, optionally can further comprise a quantity, generally not more than 50% of the SU content of the composition, of one or more Other SU. As used herein “Other SU” refers to those SU A.I.s listed as “Other” in Table[4], along with their agronomically acceptable salts and esters, and combinations thereof.

In some embodiments, the Other SU content of the herbicide composition can be 50% or less by weight (wt. %) of the SU content of the composition, or about or less than 45%, 40%, 35%, 30%, 25%, 20%, 15%, 10%, 5%, 3%, 2%, or 1% by weight thereof; and, within that range, can be: 0 wt. % or more of the SU content of the composition, or about or more than 1%, 2%, 3%, 5%, 10%, 15%, 20%, 25%, 30%, 35%, 40%, or 45% by weight thereof.

In some embodiments, the Other SU content of the herbicide composition can be no more than a “substantial” amount, i.e., in this context meaning less than 50 wt. % of the SU content of the composition, e.g., from about 35 wt. % to less than 50 wt. %.

In some embodiments, the Other SU content of the herbicide composition can be no more than a “moderate” amount, i.e., in this context meaning about or less than 35 wt. % of the SU content of the composition, e.g., from about 20 wt. % to about 35 wt. %.

In some embodiments, the Other SU content of the herbicide composition can be no more than a “small” amount, i.e., in this context meaning about or less than 20 wt. % of the SU content of the composition, e.g., from about 10 wt. % to about 20 wt. %.

In some embodiments, the Other SU content of the herbicide composition can be no more than a “minor” amount, i.e., in this context meaning about or less than 10 wt. % of the SU content of the composition, e.g., from about 5 wt. % to about 10 wt. %.

In some embodiments, the Other SU content of the herbicide composition can be no more than a “minimal” amount, i.e., in this context meaning about or less than 5 wt. % of the SU content of the composition, e.g., from about 3 wt. % to about 5 wt. %.

In some embodiments, the Other SU content of the herbicide composition can be no more than a “significant” amount, i.e., in this context meaning about or less than 3 wt. % of the SU content of the composition, e.g., from about 1 wt. % to about 3 wt. %.

In some embodiments, the Other SU content of the herbicide composition can be no more than a “trace” amount, i.e., in this context meaning about or less than 1 wt. % of the SU content of the composition, e.g., from about 1 wt. % to greater than 0 wt. %.

In some embodiments, the Other SU content of the herbicide composition can be 0 wt % or can be about 0 wt. %, e.g., from about 0.5 wt. % to 0 wt. %.

In the above list of ranges of Other SU content of the herbicide composition, in those jurisdictions in which the term “about” is impermissible, this list of ranges is to be read without said term. In any remaining portion of the Description in which the term “about” is used, the Description is to be read without said term in those jurisdictions in which the term “about” is impermissible.

Optional A.I.s of other herbicide classes include ACCase inhibitors, PPO inhibitors, EPSPS inhibitors, glutamine synthetase inhibitors, p-hydroxyphenylpyruvate dioxygenase (4-HPD) inhibitors. Optional A.I.s of other types include, but are not limited to fungicides such as strobilurins, e.g., pyraclostrobin; insecticides such as nematicides, lepidoptericides, coleoptericides; molluskicides, and others known in the art.

The herbicidal compositions comprising a herbicide selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters can also comprise auxiliaries which are customary for the formulation of crop protection agents.

Examples of auxiliaries customary for the formulation of crop protection agents include inert auxiliaries, solid carriers, surfactants (such as dispersants, protective colloids, emulsifiers, wetting agents and tackifiers), organic and inorganic thickeners, penetrants (such as penetration-enhancing organosilicone surfactants or acidic sulfate chelates, e.g., CT-301™ available from Cheltec, Inc.), safeners, bactericides, antifreeze agents, antifoams, colorants, and adhesives. Formulations of the herbicide compositions useful herein can be prepared according to any method known useful therefor in the art

Examples of thickeners (i.e., compounds which impart to the formulation modified flow properties, i.e., high viscosity in the state of rest and low viscosity in motion) are polysaccharides, such as xanthan gum (Kelzan® from Kelco), Rhodopol® 23 (Rhone Poulenc) or Veegum® (from R.T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulations. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants include members of colorant classes such as the sparingly water-soluble pigments and the water-soluble dyes. Some specific examples of these include the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Suitable carriers include liquid and solid carriers.

Liquid carriers include e.g. non-aqueous solvents such as cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyc-lohexanol, ketones such as cyclohexanone, strongly polar solvents, e.g. amines such as N-methylpyrrolidone, and water as well as mixtures thereof.

Solid carriers include e.g. mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g. methylcellulose), hydrophobically modified starches, polyvinyl alcohol (Mowiol types, Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone and copolymers thereof.

Powders, materials for broadcasting and dusts can be prepared by mixing or concomitant grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water.

To prepare emulsions, pastes or oil dispersions, the herbicidal compositions comprising a herbicide selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, either as such or dissolved in an oil or solvent, can be homogenized in water by means of a wetting agent, tackifier, dispersant or emulsifier. Alternatively, it is also possible to prepare concentrates comprising active compound, wetting agent, tackifier, dispersant or emulsifier and, if desired, solvent or oil, which are suitable for dilution with water.

The concentrations of the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters in the ready-to-use preparations (formulations) can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum).

In the formulation of the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters according to the present invention the active ingredients, e.g. the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, are present in suspended, emulsified or dissolved form. The formulation according to the invention can be in the form of aqueous solutions, powders, suspensions, also highly-concentrated aqueous, oily or other suspensions or dispersions, aqueous emulsions, aqueous microemulsions, aqueous suspo-emulsions, oil dispersions, pastes, dusts, materials for spreading or granules.

The herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters according to the present invention can, for example, be formulated as follows:

1. Products for Dilution with Water

A Water-Soluble Concentrates

10 parts by weight of active compound are dissolved in 90 parts by weight of water or a water-soluble solvent. As an alternative, wetting agent(s) or other adjuvants are added. The active compound dissolves upon dilution with water. This gives a formulation with an active compound content of 10% by weight.

B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

C Emulsifiable Concentrates

15 parts by weight of active compound are dissolved in 75 parts by weight of an organic solvent (e.g. alkylaromatics) with addition of calcium dodecylbenzenesulfonate and castor oil ethoxylate (in each case 5 parts by weight). Dilution with water gives an emulsion. The formulation has an active compound content of 15% by weight.

D Emulsions

25 parts by weight of active compound are dissolved in 35 parts by weight of an organic solvent (e.g. alkylaromatics) with addition of calcium dodecylbenzenesulfonate and castor oil ethoxylate (in each case 5 parts by weight). This mixture is introduced into 30 parts by weight of water by means of an emulsifier (Ultraturrax) and made into a homogeneous emulsion. Dilution with water gives an emulsion. The formulation has an active compound content of 25% by weight.

E Suspensions

In an agitated ball mill, 20 parts by weight of active compound are comminuted with addition of 10 parts by weight of dispersants and wetting agent(s) and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules

50 parts by weight of active compound are ground finely with addition of 50 parts by weight of dispersants and wetting agent(s) and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders

75 parts by weight of active compound are ground in a rotor-stator mill with addition of 25 parts by weight of dispersants, wetting agent(s) and silica gel. Dilution with water gives a stable dispersion or solution of the active compound. The active compound content of the formulation is 75% by weight.

H Gel formulations

In a ball mill, 20 parts by weight of active compound, 10 parts by weight of dispersant, 1 part by weight of gelling agent and 70 parts by weight of water or of an organic solvent are mixed to give a fine suspension. Dilution with water gives a stable suspension with active compound content of 20% by weight.

2. Products to be Applied Undiluted

I Dusts

5 parts by weight of active compound are ground finely and mixed intimately with 95 parts by weight of finely divided kaolin. This gives a dusting powder with an active compound content of 5% by weight.

J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

K ULV Solutions (UL)

10 parts by weight of active compound are dissolved in 90 parts by weight of an organic solvent, for example xylene. This gives a product to be applied undiluted with an active compound content of 10% by weight.

Aqueous use forms can be prepared from emulsion concentrates, suspensions, pastes, wettable powders or water-dispersible granules by adding water.

The herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters or the herbicidal compositions comprising them can be applied pre-, post-emergence or pre-plant, or together with the seed of the imidazolinone-resistant winter oilseed rape plant. It is also possible to apply the herbicidal composition or active compounds by applying seed, pretreated with the herbicidal compositions or active compounds, of a crop plant.

In a further embodiment, the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters or the herbicidal compositions can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters according to the invention or the compositions prepared therefrom. Here, the herbicidal compositions can be applied diluted or undiluted.

The term “seed” comprises seed of all types, such as, for example, corns, seeds, fruits, tubers, seedlings and similar forms. Here, preferably, the term seed describes corns and seeds.

The seed used can be seed of the useful plants mentioned above, but also the seed of transgenic plants or plants obtained by customary breeding methods.

To widen the spectrum of action, the SU herbicide A.I.(s) selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, can be mixed with a large number of representatives of other herbicidal or growth-regulating active ingredient groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexane-diones, hetaryl aryl ketones, benzylisoxazolidinones, meta-CF3-phenyl derivatives, carbamates, quinolinecarboxylic acid and its derivatives, chloroacetanilides, cyclohexenone oxime ether derivatives, diazines, dichloropropionic acid and its derivatives, dihydro-benzofurans, dihydrofuran-3-ones, dinitroanilines, dinitrophenols, diphenyl ethers, dipyridyls, halocarboxylic acids and their derivatives, ureas, 3 phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils, phenyl pyrazolines and isoxazolines and derivatives thereof.

It may furthermore be beneficial to apply the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates can also be added.

Moreover, it may be useful to apply the herbicides selected from the group of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, in combination with safeners. Safeners are chemical compounds which prevent or reduce herbicide-induced injury to useful plants without having a major impact on the herbicidal action of the herbicides amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, towards unwanted plants. They can be applied either before sowings (e.g. on seed treatments, shoots or seedlings) or in the pre-emergence application or post-emergence application of the useful plant. The safeners and the herbicides amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and optionally other agronomic A.I.(s), e.g., one or more imidazolinones selected from the group of imazamox, imazethapyr, imazapyr, imazapic, combinations thereof, and their agriculturally suitable salts and esters, can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenyl-carbamates and their agriculturally acceptable salts and their agriculturally acceptable derivatives such amides, esters, and thioesters, provided they have an acid group.

Methods of Controlling Weeds

Herbicide-tolerant plants of the invention can be used in conjunction with an herbicide to which they are tolerant. Herbicides can be applied to the plants of the invention using any techniques known to those skilled in the art. Herbicides can be applied at any point in the plant cultivation process. For example, herbicides can be applied pre-planting, at planting, pre-emergence, post-emergence or combinations thereof.

Herbicide compositions hereof can be applied, e.g., as foliar treatments, soil treatments, seed treatments, or soil drenches. Application can be made, e.g., by spraying, dusting, broadcasting, or any other mode known useful in the art.

In one embodiment, herbicides can be used to control the growth of weeds that may be found growing in the vicinity of the herbicide-tolerant plants invention. In embodiments of this type, an herbicide can be applied to a plot in which herbicide-tolerant plants of the invention are growing in vicinity to weeds. An herbicide to which the herbicide-tolerant plant of the invention is tolerant can then be applied to the plot at a concentration sufficient to kill or inhibit the growth of the weed. Concentrations of herbicide sufficient to kill or inhibit the growth of weeds are known in the art and are disclosed above.

The methods of controlling weeds can also include a step of selecting a winter-type Brassica plant capable of tolerating the SU herbicide composition. As used herein, a step of selecting a winter-type Brassica plant capable of tolerating the SU herbicide composition can be performed by a person's choosing the plant to be grown, or by a first person's choosing to have a second person choose the plant to be grown. For example, a Brassica producer may be operating as a seed multiplier to produce seed, or operating as a grain grower to produce grain or other produce for market. In either situation, the Brassica producer can choose for himself what Brassica variety to grow, or can permit another to choose what Brassica variety he will grow, or can have pre-chosen by prior contractual arrangement to grow a Brassica variety to be chosen by a third party, e.g., pursuant to a service agreement, a forward contract, or other arrangement. All such modes by which a Brassica producer chooses what Brassica variety to grow can constitute a step of selecting a winter-type Brassica plant hereof.

Methods of Providing Yield Protection

Methods of planting, growing and treating with SU herbicide compositions winter-type Brassica plants according to various embodiments of the present invention can provide yield protection to a winter-type Brassica crop grown in the presence of a sulfonylurea (SU) herbicide composition. The methods can comprise:
planting a seed of a winter-type Brassica plant in the presence of the SU herbicide composition; and
growing the seed under conditions which will produce the winter-type Brassica plant;
wherein said Brassica plant comprises at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica;

wherein the yield can be equal to or greater than that provided by a wild-type version of the same type of winter-type Brassica plant.

These methods can also comprise:
planting a seed of a winter-type Brassica plant;
growing the seed under conditions which will produce the winter-type Brassica plant; and
performing an herbicide treatment of the plant by applying an herbicide composition, comprising sulfonylurea(s) (SU), to the plant and its immediate vicinity; wherein said Brassica plant comprises at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica;
   wherein the yield can be equal to or greater than that provided by a wild-type version of the same type of winter-type Brassica plant. The herbicide treatment may be a pre-flowering treatment of the plant.

The methods of providing yield protection can also include a step of choosing a winter-type Brassica plant capable of tolerating the SU herbicide composition.

The methods of providing yield protection can further comprise harvesting seeds produced by the winter-type Brassica plants. These methods can also control weeds in the vicinity of the winter-type Brassica plants.

As used herein, “yield protection” includes, but is not limited to, a reduced risk of crop loss, reduction in yield or both. Negative effects of SU herbicide compositions on winter-type Brassica plants can include, but are not limited to, death; transient plant injury; delayed growth; altered maturation; significant visual injury symptoms; decreases in field plant density (i.e., fewer plants in the population); and increases in the proportion of plants exhibiting delayed-maturation, smaller stature (less biomass) and/or injury from disease or insect attack beginning during periods of temporary metabolic stress/wilt phase (i.e., transitory SU herbicide injury). Non-SU-tolerant winter-type Brassica plants typically suffer from negative effects in the presence of SU herbicide compositions; and winter-type Brassica plants that are tolerant solely to residual amounts of SU herbicides can also suffer from negative effects. However, winter-type Brassica plants of the invention are more robust to exposure to such SU herbicide compositions. Thus, a winter-type Brassica crop grown from SU-tolerant winter-type Brassica plants of the invention can provide a greater yield than such SU herbicide susceptible or residual tolerant plants grown in the presence of SU herbicide compositions.

A number of desires can motivate the step of choosing a winter-type Brassica plant capable of tolerating SU herbicide compositions. For example, and without limitation, a Brassica producer may desire: (1) to control Brassica crop weeds using an SU application, wherein an SU herbicide application could not otherwise be applied to such Brassica crop without substantial crop injury or loss; or (2) to avoid or decrease the risk of permanent or transient crop injury from SU residues in soil, or to avoid or decrease such risk better than can use of a Brassica that is only SU-residue-tolerant; or (3) to avoid or decrease the risk of permanent or transient crop injury from SU residues present in tanks re-used for preparing or supplying other agronomic products to the crop, or to avoid or decrease such risk better than can use of a Brassica that is only SU-residue-tolerant. Choosing winter-type Brassica plants of the invention can achieve these goals.

In some embodiments, a Brassica producer may desire to control Brassica crop weeds using an SU application. Among the Brassica crop weeds that can be treated in various embodiments hereof are, e.g., Brassicaceae family weeds such as Wild turnip (Brassica tournefortii), Shepherd's purse (Capsella bursa-pastoris), Hare's ear mustard (Conringia orientalis), Wormseed mustard (Erysimum cheiranthoides; Treacle mustard), Buchan weed (Hirschfeldia incana), Common peppergrass (Lepidium virginicum; Virginia pepperweed), Musk weed (Myagrum perfoliatum), Ball mustard (Neslia paniculata), Wild radish (Raphanus raphanistrum), Turnip weed (Rapistrum rugosum), Wild mustard (Sinapis arvensis; Charlock), Indian hedge mustard (Sisymbrium orientate), Flixweed (Sisymbrium sophia; Tansy mustard; Fluxweed), and Stinkweed (Thlaspi arvense; Field pennycress). Such Brassicacea family weeds can be problematic to control in traditional Brassica crops. Choosing winter-type Brassica plants of the invention can achieve these goals.

As used herein, a step of choosing a winter-type Brassica plant capable of tolerating the SU herbicide composition can be performed by a person's choosing the plant to be grown, or by a first person's choosing to have a second person choose the plant to be grown. For example, a Brassica producer may be operating as a seed multiplier to produce seed, or operating as a grain grower to produce grain or other produce for market. In either situation, the Brassica producer can choose for himself what Brassica variety to grow, or can permit another to choose what Brassica variety he will grow, or can have pre-chosen by prior contractual arrangement to grow a Brassica variety to be chosen by a third party, e.g., pursuant to a service agreement, a forward contract, or other arrangement. All such modes by which a Brassica producer chooses what Brassica variety to grow can constitute a step of choosing a winter-type Brassica plant hereof.

A crop of the winter-type Brassica plants of the invention grown in soil containing SU herbicides, and optionally containing sulfonamide and/or imidazolinone AHAS inhibiting herbicides, can achieve a higher yield than a crop of winter-type Brassica plants of the corresponding wild-type isoline grown in the same herbicide containing conditions. Additionally, a crop grown of the winter-type Brassica plants of the invention and exposed to SU herbicides from sprayers contaminated with SU herbicides from leftovers in herbicide mixing tanks provide a yield protection benefit compared to winter-type Brassica plants of the corresponding wild-type isoline.

Crops of winter-type Brassica plants of the invention can produce substantially equivalent yields when exposed to SU herbicides and when not exposed to SU herbicides, when grown under otherwise similar conditions. Additionally, crops of winter-type Brassica plants of the invention can achieve equal yields when exposed to SU herbicides from contaminated sprayers and when not exposed to SU herbicides from contaminated sprayers, when grown under otherwise similar conditions.

Winter-type Brassica Crop Containing AHASL Gene Encoding Mutation at P197(At) or W574(At) and Located in Any Brassica Genome.

The present invention also relates to winter-type Brassica plants having an expressible plastidic AHASL gene that encodes a mutation at P197(At) or W574(At) located in any genome, for example a Brassica A-, B- or C-genome. This is an exception to the above-described embodiments where the herbicide-tolerant AHASL gene can be located only in the Brassica A genome. In those embodiments in which the WOSR or other winter-type Brassica crop contains an expressible plastidic AHASL gene that encodes a mutation at P197(At) or W574(At), if that mutation is encoded in a Brassica B- or C-genome allele, at least one additional mutation must also be encoded in the plant, in the same or different expressible plastidic AHASL gene, where that mutation is selected from those substitutions at sites: G121(At), A122(At), M124(At), R142(At), V196(At), R199(At), T203(At), A205(At), F206(At), K256(At), M351(At), H352(At), R373(At), D375(At), D376(At), R377(At), M570(At), V571(At), F578(At), S653(At), and G654(At); and preferably at sites from among A122(At), R199(At), A205(At), S653(At), and G654(At); and more preferably at S653(At). Winter-type Brassica plants of this embodiment can be employed in the methods described throughout the detailed description hereof, including methods for controlling weeds with performing post-emergent herbicide treatment, methods for selecting plants and methods for providing yield protection.

The present invention provides a method for controlling weeds in a winter-type Brassica crop including the steps of: performing post-emergent treatment of an herbicide-tolerant (HT) Brassica plant of said crop by applying an herbicide composition to the plant and its immediate vicinity, at a dose rate in the range from 0.25× to about 4× of SU, wherein said herbicide composition comprises a SU; and said Brassica plant (1) comprises at least one herbicide tolerant AHASL (HT-AHASL) gene, wherein one of the HT-AHASL genes encodes a sulfonylurea tolerance HT (SU-HT) mutation selected from P197X and W574X, and at least one additional mutation selected from G121X, A122X, M124X, V196X, R199X, T203X, A205X, F206X, K256X, M351X, H352X, R373X, D375X, D376X, R377X, M570X, V571X, F578X, S653X, and G654X; or (2) comprises at least two herbicide tolerant AHASL (HT-AHASL) genes wherein a first HT-AHASL gene encodes a sulfonylurea tolerance (SU-HT) mutation selected from P197X and W574X and a second HT-AHASL gene encodes a mutation selected from G121X, A122X, M124X, V196X, R199X, T203X, A205X, F206X, K256X, M351X, H352X, R373X, D375X, D376X, R377X, M570X, V571X, F578X, S653X, and G654X.

In some embodiments, said herbicide composition can comprise other agronomically useful fauns of sulfonylurea(s). In some embodiments, the sulfonylurea is selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof. In some embodiments, the herbicide composition comprises a significant amount of no other SU.

In some embodiments said SU-HT mutation is selected from P197A, P197S, P197L, and W574L. In some embodiments, only one of the HT-AHASL genes encodes the SU-HT mutation selected from P197X and W574X. In some embodiments, said at least one additional mutation is selected from A122T, A122Q, A122V, P197L, P197A, P197S, A205V, R199A, A205V, W574L, S653N, G654E, and G654D. In some embodiments, said at least one additional mutation is selected from A122T, R199A, A205V, G654E, and S653N.

It will be readily apparent to one of ordinary skill in the relevant arts that other suitable modifications and adaptations to the methods and applications described herein are obvious and can be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

Specific examples of the preparation of herbicide-tolerant plants of the invention are provided below.

As used herein, “no significant” plant injury equates to injuries with a score of 5 or less on the 0-100 PHYTOX scale, described below, preferably injuries having a score of 4, 3, 2, 1, or less. In some embodiments, “no significant” plant injury can be a transient injury lasting 5 days or fewer, and preferably lasting 4, 3, 2, or 1 day or less.

Abbreviations and acronyms used herein are defined as follows:

ED₅₀=effective dose (i.e., dose required to produce a desired effect in 50% of a population);
DAT=days after treatment;
DALT=days after last treatment;
DAP=days after planting;
GS=growth stage
BBCH=industry-recognized standard for identifying phenological stages of growth in canola

### Example 1

Herbicide tolerance in AHAS-inhibitor tolerant WOSR plant lines compared to AHAS-inhibitor susceptible (conventional) WOSR plant lines.

Plants of an AHAS-inhibitor tolerant WOSR line, for example a line a representative seed sample of which is deposited under ATCC Deposit No. 40684, and plants of a first AHAS-inhibitor susceptible WOSR line are sown in 10 cm pots with a sandy loam. Each pot is planted with two plants, the plants are watered from beneath and fertilized according there requirement. The pots are stored side by side in a greenhouse at 12° C. at the emergence phase. The temperature increases to 15-20° C. three weeks after sowing.

A post-emergence treatment of herbicides is applied to the plants by means of fine distributed nozzles and a water use rate of 200 L/ha at the growth stage GS/BBCH 12 (2 true leaf stage). The herbicides are tested at different rates. Five cultivar replicates are carried out per rate.

The evaluation of efficacy is assessed as crop damage caused by the herbicides using a scale from 0 to 100%, compared to the untreated control plants. Here, 0 means no damage and 100 means complete destruction of the plants. The level of efficacy is assessed 21-22 days after treatment (DAT). The efficacy results are presented as ED₅₀ values.

TABLE 5

| | Plants of first | Plants of |
|---|---|---|
| | AHAS-inhibitor | AHAS-inhibitor |
| | susceptible | tolerant |
| | WOSR line | WOSR line |
| AI | ED₅₀ (g/ai/ha) | ED₅₀ (g/ai/ha) |
| Tribenuron-methyl | 2.59 | 25.88 |
| Florasulam | 1.51 | 6.53 |
| Flupyrsulfuron-methyl-sodium | 0.58 | 63316.17 |
| Metsulfuron-methyl | 0.57 | 5.06 |
| Tritosulfuron | 0.39 | 42.30 |
| Sulfosulfuron | 1.70 | 2417.06 |
| Propoxycarbazone-sodium | 2.16 | 67.43 |
| Iodosulfuron Mesosulfuron | 0.27 | 11.31 |
| Chlorsulfuron | 0.42 | 2.8 |
| Trisulfuron-methyl 500 | 3.36 | 41.81 |

### Example 2

Herbicide tolerance in AHAS-inhibitor tolerant WOSR plant lines compared to AHAS-inhibitor susceptible (conventional) WOSR plant lines.

Plants of an AHAS-inhibitor tolerant WOSR line and plants of a second AHAS-inhibitor susceptible WOSR line are sown in 10 cm pots with a sandy loam. Each pot is planted with two plants, the plants are watered from beneath and fertilized according there requirement. The pots are stored side by side in a greenhouse at 12° C. at the emergence phase. The temperature increases to 15-20° C. three weeks after sowing.

A post-emergence treatment of herbicides is applied to the plants by means of fine distributed nozzles and a water use rate of 200 L/ha at the growth stage GS/BBCH 10. The herbicides are tested at different rates. Twelve cultivar replicates are carried out per rate.

The evaluation of efficacy is assessed as crop damage caused by the herbicides using a scale from 0 to 100%, compared to the untreated control plants. Here, 0 means no damage and 100 means complete destruction of the plants. The level of efficacy is assessed 19 days after treatment (DAT). The efficacy results are presented as ED₅₀ values.

TABLE 6

| | Plants of second | Plants of |
|---|---|---|
| | AHAS-inhibitor | AHAS-inhibitor |
| | susceptible | tolerant |
| | WOSR line | WOSR line |
| AI | ED₅₀ (g/ai/ha) | ED₅₀ (g/ai/ha) |
| Tribenuron-methyl | 0.11 | 9.11 |
| Florasulam | 0.28 | 4.9 |
| Flupyrsulfuron-methyl-sodium | 0.79 | 147.97 |
| Metsulfuron-methyl | 0.17 | 2.28 |
| Tritosulfuron | 1.76 | 2.96 |
| Sulfosulfuron | 0.01 | 3.29 |
| Propoxycarbazone-sodium | 0.17 | 42.07 |
| Iodosulfuron Mesosulfuron | 0.07 | 0.62 |
| Chlorsulfuron | 0.04 | 11.2 |
| Trisulfuron-methyl 500 | 0.03 | 4.48 |

### Example 3

Enhanced herbicide tolerance in plants of AHAS-tolerant WOSR plant lines compared to plants of AHAS-tolerant spring oilseed rape (SOSR) plant lines.

Plants of an AHAS-inhibitor tolerant WOSR line and plants of an AHAS-tolerant SOSR line are sown side by side in a plot. A post-emergence treatment of herbicides is applied to the plants by means of fine distributed nozzles at the growth stage GS/BBCH 12/13. There are four replications of each treatment.

The evaluation for crop-tolerance is assessed as PHYTOX symptom caused by the chemical compounds carried out using a scale from 0 to 100%, compared to the untreated control plants. Here, 0 means no damage and 100 means complete destruction of the plants.

TABLE 7

| | | | Crop Variety | |
|---|---|---|---|---|
| | | | WOSR | SOSR |
| | | | Crop GS from/to/method | |
| | | | 14/15/B | 13/14/B |
| | | | DAT/DALT/DAP | |
| | Product | AI | 15/15/37 | 15/15/37 |
| | Rate | Rate | Eval. Unit | |
| AI | (kg/ha) | (g/ha) | PHYTOX % | PHYTOX % |
| Flupyrsulfuron- | 0.01 | 5 | 11 | 19 |
| methyl-sodium | | | | |
| Thifensulfuron- | 0.03 | 15 | 13 | 23 |
| methyl | | | | |
| Tritosulfuron | 0.035 | 25 | 43 | 53 |
| Tribenuron-methyl | 0.03 | 15 | 17 | 25 |

### Example 4

Enhanced herbicide tolerance in plants of AHAS-inhibitor tolerant WOSR plant lines compared to plants of AHAS-inhibitor susceptible (conventional) WOSR plant lines.

Plants of an AHAS-inhibitor tolerant WOSR line and plants of an AHAS-inhibitor susceptible WOSR line are sown side by side in a plot. A pre-emergence treatment of herbicides is applied to the plants by means of fine distributed nozzles. There are four replications of each treatment.

The evaluation for crop-tolerance is assessed as PHYTOX symptom caused by the chemical compounds carried out using a scale from 0 to 100%, compared to the untreated control plants. Here, 0 means no damage and 100 means complete destruction of the plants.

TABLE 8

| | | | Resistance/Variety | |
|---|---|---|---|---|
| | | | AHAS- | AHAS- |
| | | | inhibitor | inhibitor |
| | | | tolerant | tolerant |
| | | | WOSR line | WOSR line |
| | | | Crop GS from/to | |
| | | | 10/11 | 11/13 |
| | | | DAT/DALT/DAP | |
| | Product | AI | 10/10/10 | 29/29/29 |
| | Rate | Rate | Eval. Unit | |
| AI | (g/ha) | (g/ha) | PHYTOX % | PHYTOX % |
| Propoxycarbazone | 10 | 7 | 0 | 0 |
| Sulfosulfuron | 2.5 | 2 | 0 | 0 |
| Flupyrsulfuron- | 50 | 1.8 | 0 | 0 |
| methyl | | | | |
| Amidosulfuron | 20 | 2.75 | 0 | 0 |

TABLE 9

| | | | Resistance/Variety | |
|---|---|---|---|---|
| | | | AHAS- | AHAS- |
| | | | inhibitor | inhibitor |
| | | | susceptible | susceptible |
| | | | WOSR line | WOSR line |
| | | | Crop GS from/to | |
| | | | 10/11 | 11/13 |
| | | | DAT/DALT/DAP | |
| | Product | AI | 10/10/10 | 29/29/29 |
| | Rate | Rate | Eval. Unit | |
| AI | (g/ha) | (g/ha) | PHYTOX % | PHYTOX % |
| Propoxycarbazone | 10 | 7 | 100 | 100 |
| Sulfosulfuron | 2.5 | 2 | 99 | 99 |
| Flupyrsulfuron- | 50 | 1.8 | 100 | 100 |
| methyl | | | | |
| Amidosulfuron | 20 | 2.75 | 100 | 100 |

### Example 5

### AHAS Activity in the Presence of Imidazolinone Herbicides

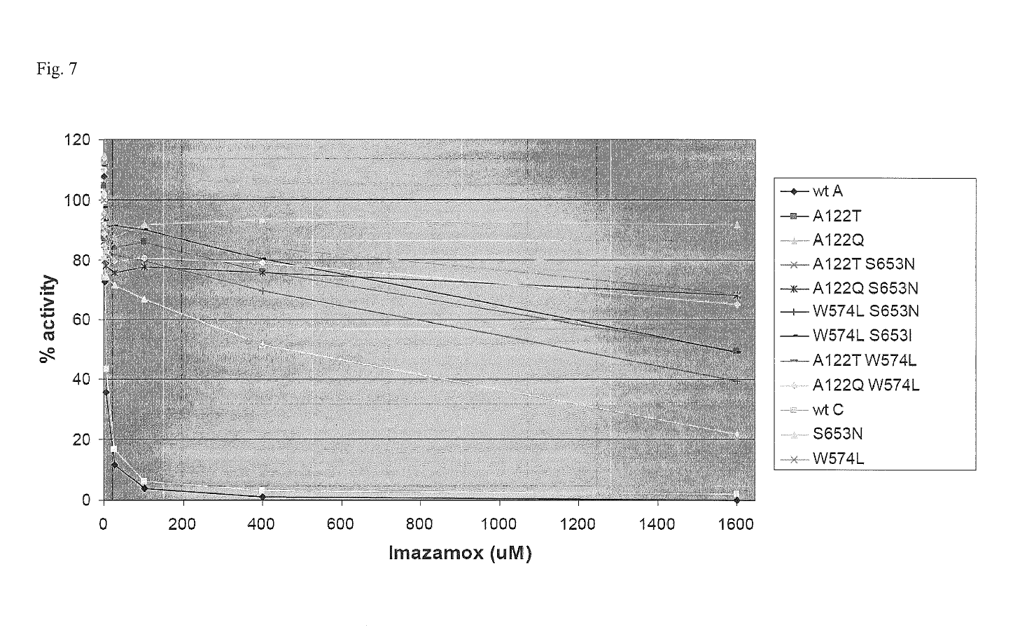

AHAS enzymes with various mutations are reacted with pyruvate and treated with water and serial solutions of varying imazamox concentration to determine AHAS activity. Reactions proceed at 37° C. for 45 minutes and are terminated by addition of 20 μL of a solution of 5% sulfuric acid with heating at 60° C. for 15-30 minutes to convert acetolactate to acetoin. The resulting acetoin is incubated with creatin and naphthyl (creatin-naphthyl complex) in sodium hydroxide solution at 60° C. for 15 minutes to produce a colored product for measurement and correlation with activity of the AHAS enzymes. FIG. 7 shows the AHAS enzyme activity.

### Example 6

### AHAS Activity in the Presence of Sulfonylurea Herbicides

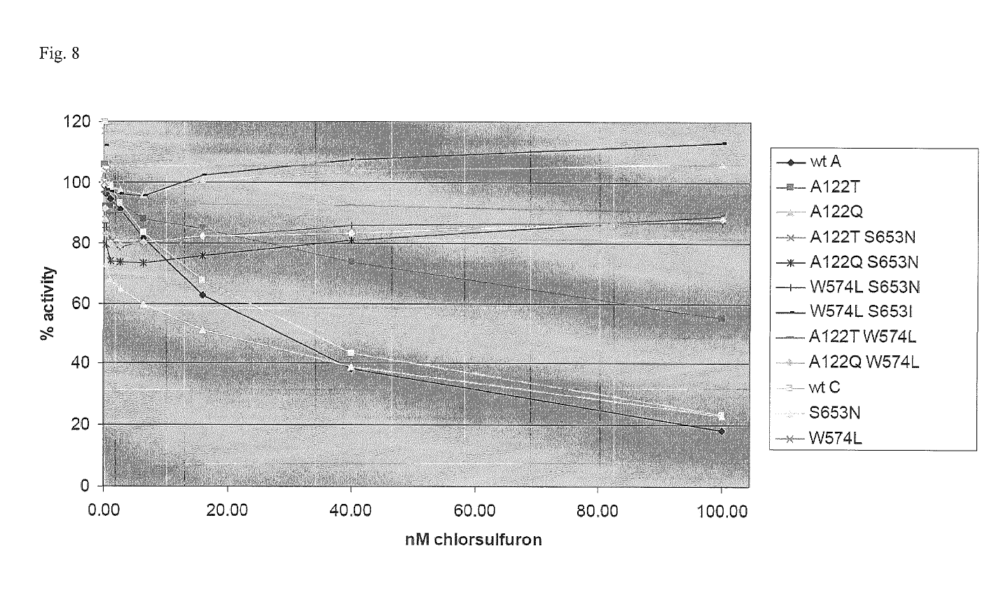

AHAS enzymes with various mutations are reacted with pyruvate and treated with water and serial solutions of varying chlorsulfuron concentration to determine AHAS activity. Reactions proceed at 37° C. for 45 minutes and are terminated by addition of 20 μL of a solution of 5% sulfuric acid with heating at 60° C. for 15-30 minutes to convert acetolactate to acetoin. The resulting acetoin is incubated with creatin and naphthyl (creatin-naphthyl complex) in sodium hydroxide solution at 60° C. for 15 minutes to produce a colored product for measurement and correlation with activity of the AHAS enzymes. FIG. 8 shows the AHAS enzyme activity.

While the foregoing invention has been described in some detail for purposes of clarity and understanding, it will be appreciated by one skilled in the art from a reading of this disclosure that various changes in form and detail can be made without departing from the true scope of the invention and appended claims. All patents and publications cited herein are entirely incorporated herein by reference.

1. A method for controlling weeds in a winter-type Brassica crop comprising:
performing treatment of an herbicide-tolerant (HT) Brassica plant of said crop by applying an herbicide composition, comprising sulfonylurea(s) (SU), wherein
(A) said treatment is:
(1) a post-emergent treatment wherein the herbicide composition is applied to the plant and its immediate vicinity at a dose rate in the range of from 0.25× to about 4× of SU and the herbicide composition:
(a) comprises a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and
(b) comprises not more than a significant amount of any other SU; or
(2) a pre-emergent treatment, or 0 to 30 days pre-planting treatment, wherein the herbicide composition is applied to the seed planting locus and its immediate vicinity at a dose rate in the range from greater than 0.5× to about 4× of SU and the herbicide composition:
(a) comprises a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and
(b) comprises a significant amount of no other SU; and
(B) said Brassica plant comprises at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica plant; and wherein said Brassica plant is capable of tolerating said SU treatment at a dose rate of 1× of SU with no significant SU-herbicide-induced injury from said treatment.

2. (canceled)

3. The method of claim 1 further comprising choosing, prior to treatment with the herbicide composition, said herbicide tolerant Brassica plant.

4. The method of claim 1 further comprising choosing seed capable of producing said herbicide tolerant Brassica plant, planting said seed and growing said herbicide tolerant Brassica plant from said seed.

5. The method of claim 1 wherein said mono-SU-HT-AHASL gene:
(a) encodes only one of P197X or W574X and encodes it homozygously or hemizygously;
(b) encodes only one of P197X or W574X and encodes it in one allele and encodes a wild-type P197P or W574W residue, respectively, in the homologous allele;
(c) encodes two different P197X mutations heterozygously and is homozygous for wild-type W574W residues;
(d) encodes two different W574X mutations heterozygously and is homozygous for wild-type P197P residues; or
(e) encodes both (i) one P197X mutation and a wild-type W574W residue in one allele, and (ii) one W574X mutation and a wild-type P197P residue in the homologous allele

6. A method for selecting an HT winter-type Brassica plant comprising:
(I) performing post-emergent treatment of Brassica plants by applying an herbicide composition, comprising sulfonylurea(s) (SU), to the plant and its immediate vicinity, at a dose rate in the range from 0.25× to about 4× of SU, according to items (A)(1) and (B) of claim 1; and
(II) selecting a Brassica plant capable of tolerating said post-emergent SU treatment at a dose rate of at least 1× of SU with no significant SU-herbicide-induced injury from said treatment.

7. A method for selecting an HT winter-type Brassica plant comprising:
(I) performing pre-emergent treatment, or 0 to 30 days-pre-planting treatment, of Brassica plants by applying an herbicide composition, comprising sulfonylurea(s) (SU), to the seed planting locus thereof and its immediate vicinity, at a dose rate in the range from greater than 0.5× to about 4× of SU, according to items (A)(2) and (B) of claim 1; and
(II) selecting a Brassica plant capable of tolerating said post-emergent SU treatment at a dose rate of 1× of SU with no significant SU-herbicide-induced injury from said treatment.

8. A method of providing yield protection for a winter-type Brassica crop grown in the presence of a sulfonylurea (SU) herbicide composition comprising:
planting a seed of a winter-type Brassica plant in the presence of the SU herbicide composition or performing an herbicide treatment of the plant by applying an herbicide composition, comprising sulfonylurea(s) (SU), to the plant and its immediate vicinity; and
growing the seed under conditions capable of producing the winter-type Brassica plant;
wherein said Brassica plant comprises at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica plant; and
wherein the yield is equal to or greater than the yield provided by a wild-type version of the same type of winter-type Brassica plant.

9. (canceled)

10. The method of claim 8 further comprising choosing, prior to the herbicide treatment, said seed of said winter-type Brassica plant.

11. The method according to claim 1 or 8, wherein said herbicide composition further comprises one or more imidazolinone herbicides.

12. The method according to claim 11, wherein the imidazolinone is imazamox or an agronomically acceptable salt or ester thereof.

13. The method according to claim 1 or 8, wherein the herbicide composition comprises a combination of iodosulfuron and mesosulfuron.

14. The method of claim 1 or 8 wherein the SU-HT mutation is selected from the group consisting of P197S, P197A, P197E, P197L, P197Q, P197R, P197S, P197V, P197W, P197Y, P1971, P197H, P197C, P197G, W574L, W574M, W574C, W574S, W574R, W574G, W574A, W574F, W574Q, and W574Y.

15. The method of claim 14 wherein the SU-HT mutation is selected from the group consisting of P197S, P197L, P197T, and W574L.

16. (canceled)

17. (canceled)

18. The method according to claim 1 or 8, wherein the Brassica plant comprises an Other HT-mutation selected from the group consisting of A122X, R199X, A205X, S653X, G654X, and combinations thereof.

19. The method according to claim 18, wherein the Brassica plant comprises an Other HT-mutation selected from the group consisting of A122T, A122V, A122D, A122P, A122Y, R199A, R199E, A205V, A205C, A205D, A205E, A205R, A205T, A205W, A205Y, A205N, S653N, S653I, S653F, S653T, G654Q, G654C, G654E, G654D, and combinations thereof.

20. The method according to claim 1, wherein the Brassica plant comprises one HT-AHASL/197/574 gene, whose SU-HT mutation is W574X.

21. The method according to claim 20, wherein said Brassica plant further comprises at least one Other HT-mutation.

22. The method according to claim 21, wherein the Other HT-mutation selected from the group consisting of A122X, R199X, A205X, S653X, G654X, and combinations thereof.

23. The method according to claim 22, wherein the Other HT-mutation is selected from the group consisting of A122T, A122V, R199A, R199E, A205V, S653N, G654E, and combinations thereof.

24. (canceled)

25. (canceled)

26. The method according to claim 21, wherein the Other HT-mutation is encoded by an HT-AHASL gene located in the Brassica A-genome of the plant.

27. The method according to claim 21, wherein the Other HT-mutation is encoded by the mono-SU-AHASL gene.

28. The method according to claim 8 further comprising harvesting seed produced by the winter-type Brassica plant.

29. The method according to claim 8 wherein the SU herbicide composition comprises
(A) a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and
(B) not more than a significant amount of any other SU.

30. (canceled)

31. The method according to claim 8 wherein the SU herbicide composition is present as a soil residue of an SU herbicide composition applied to a previous crop.

32. The method according to claim 8 wherein the yield of the winter-type Brassica crop is higher than a yield of a crop of winter-type Brassica plants of the corresponding wild-type isoline and grown in the same herbicide containing conditions.

33. The method according to claim 8 wherein the yield of the winter-type Brassica crop is substantially equivalent to the yield when the winter-type Brassica crop is grown in absence of the SU herbicide composition.

34. The method of claim 10 wherein the chosen Brassica plant is capable of tolerating said herbicide treatment at a dose rate of at least 1× of SU with no significant SU-herbicide-induced injury from said treatment.

35. The method according to claim 1 or 8, wherein said herbicide composition comprises at least one further A.I. chosen from among: EPSPS-inhibitors, glyphosate; glutamine synthetase inhibitors, glufosinate; ACCase inhibitors, dims, fops, or dens; PPO-inhibitors, saflufenacil; fungicides, pyraclostrobin; or agronomically acceptable salts or esters thereof; and the Brassica plant expresses a trait of tolerance to said further A.I.(s).

36. A method for controlling weeds in a winter-type Brassica crop comprising:
performing post-emergent treatment of an herbicide-tolerant (HT) Brassica plant of said crop by applying an herbicide composition, comprising sulfonylurea(s) (SU), to the plant and its immediate vicinity, at a dose rate in the range from 0.25× to about 4× of SU, wherein
(A) said herbicide composition
(1) comprises a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and
(2) optionally comprises up to a substantial amount of total Other SU content; and
(B) said Brassica plant comprises at least one herbicide tolerant AHASL (HT-AHASL) gene, wherein only one of the HT-AHASL genes in the plant encodes a sulfonylurea herbicide tolerance (SU-HT) mutation selected from P197X and W574X and is a mono-SU-HT-AHASL gene, which can optionally encode Other HT mutation(s), and wherein said mono-SU-HT-AHASL gene is located in the A genome of said Brassica plant; and
wherein said Brassica plant is capable of tolerating said post-emergent SU treatment at a dose rate of 1× of SU with no significant SU-herbicide-induced injury from said treatment.

37. The method according to claim 36, wherein said herbicide composition (A) optionally comprises up to a moderate amount of Other SU, up to a small amount of Other SU, up to a minor amount of Other SU, up to a minimal amount of Other SU, up to a trace amount of Other SU, or about 0 wt. % of other SU.

38-42. (canceled)

43. A method for producing seed comprising performing the method of claim 1 or 8 and harvesting seed from said Brassica plant.

44. A seed harvested from the Brassica plant treated according to the method of claim 1 or 8.

45. A method for controlling weeds in a winter-type Brassica crop comprising:
performing post-emergent treatment of an herbicide-tolerant (HT) Brassica plant of said crop by applying an herbicide composition, comprising sulfonylurea(s) (SU), to the plant and its immediate vicinity, at a dose rate in the range from 0.25× to about 4× of SU, wherein
(A) said herbicide composition
(1) comprises a SU selected from the group consisting of amidosulfuron, flupyrsulfuron, foramsulfuron, imazosulfuron, iodosulfuron, mesosulfuron, nicosulfuron, thifensulfuron, and tribenuron, agronomically acceptable salts and esters thereof, and combinations thereof, and
(2) comprises a significant amount of no other SU; and
(B) said Brassica plant
(1) comprises at least one herbicide tolerant AHASL (HT-AHASL) gene, wherein one of the HT-AHASL genes encodes a sulfonylurea tolerance HT (SU-HT) mutation selected from P197X and W574X, and at least one additional mutation selected from G121X, A122X, M124X, V196X, R199X, T203X, A205X, F206X, K256X, M351X, H352X, R373X, D375X, D376X, R377X, M570X, V571X, F578X, S653X, and G654X; or
(2) comprises at least two herbicide tolerant AHASL (HT-AHASL) genes wherein a first HT-AHASL gene encodes a sulfonylurea tolerance (SU-HT) mutation selected from P197X and W574X and a second HT-AHASL gene encodes a mutation selected from G121X, A122X, M124X, V196X, R199X, T203X, A205X, F206X, K256X, M351X, H352X, R373X, D375X, D376X, R377X, M570X, V571X, F578X, S653X, and G654X;
wherein said Brassica plant is capable of tolerating said post-emergent SU treatment at a dose rate of 1× of SU with no significant SU-herbicide-induced injury from said treatment.
